(19) United States
(12) Patent Application Publication
(10) Pub. No.: US 2026/0295824 A1
(43) Pub. Date: Oct. 1, 2026

(54) CONTROLLING A SURGICAL ROBOT ARM WHILST ENTERING A SLEEP MODE

(71) Applicant: CMR Surgical Limited, Cambridge (GB)

(72) Inventors: George Ebberson, Cambridge (GB); Edward James Wildin Tucker, Cambridge (GB); Andrew Jason Penner, Cambridge (GB)

(73) Assignee: CMR Surgical Limited, Cambridge, OT (GB)

(21) Appl. No.: 18/996,520

(22) PCT Filed: Jul. 31, 2023
(86) PCT No.: PCT/GB2023/052014

§ 371 Date: Jan. 17, 2025

(30) Foreign Application Priority Data
Aug. 15, 2022 (GB) 2211900.2

(51) Int. Cl. B25J 9/16; A61B 34/20; A61B 34/37; A61B 50/13; A61B 90/00; B25J 13/08

(52) U.S. Cl. CPC B25J 9/1633; A61B 34/20; A61B 34/37; A61B 50/13; B25J 13/085; A61B 2090/031

(57) ABSTRACT

A control unit for controlling a surgical robot arm to enter a sleep mode receives a command signal to enter the sleep mode and controls, articulated links of the surgical robot arm to be driven to a compact configuration relative to a support structure. The control unit determines a collision between the surgical robot arm and the support structure from sensor data from the surgical robot arm, causes the surgical robot arm to change into a locked mode, and controls the articulated links to maintain their position. The control unit receives a command signal to enter an unlocked mode and controls the articulated links to be driven to move the surgical robot arm away from the site of the determined collision. The control unit receives a command signal to enter the sleep mode and controls the articulated links to be driven to adopt the compact configuration.

### BACKGROUND

It is known to use robots for assisting and performing surgery. FIG. 1 illustrates a typical surgical robotic system. A surgical robot 100 consists of a base 102, an arm 104 and an instrument 106. The base supports the robot and may itself be attached rigidly to a support structure, for example, the operating theatre floor, the operating theatre ceiling or a cart. The arm extends between the base and the instrument. The arm is articulated by means of multiple flexible joints 108 along its length, which are used to locate the surgical instrument in a desired location relative to the patient. The surgical instrument is attached to the distal end of the robot arm. The surgical instrument penetrates the body of the patient at a port so as to access the surgical site. The surgical instrument comprises a shaft connected to a distal end effector 110 by a jointed articulation. The end effector engages in a surgical procedure.

A surgeon controls the surgical robot 100 via a remote surgeon console 112. The surgeon console comprises one or more surgeon input devices 114. These may take the form of a hand controller or foot pedal. The surgeon console also comprises a display 116.

A control system 118 connects the surgeon console 112 to the surgical robot 100. The control system receives inputs from the surgeon input device(s) 114 and converts these to control signals to move the joints of the robot arm 104 and instrument 106. The control system sends these control signals to the robot, where the corresponding joints are driven accordingly.

FIG. 2 illustrates a known arrangement in which the surgical robot 100 is mounted on a cart 200. The robot arm is covered in a surgical drape 201 to provide a sterile barrier, thereby preventing the patient being exposed to the non-sterile robot arm and cart. A surgical instrument 106 is attached to the end of the robot arm. The robot arm is in a pose ready for inserting the instrument into the patient to perform an operation.

At the end of the operation, the instrument and drape are removed from the robot arm of FIG. 2, and the robot arm is controlled to enter a sleep mode. In this sleep mode, the robot arm is driven to fold into a predetermined compact configuration in which it can be stored. The robot is then powered off and stowed away. If the instrument or drape are not removed, or not properly removed, then the robot arm may not be able to adopt the predetermined compact configuration. The robot arm is nevertheless driven to reach this predetermined compact configuration which can lead to a collision between the robot arm and the cart. The control system detects the collision and responds by causing the robot arm to enter a fault state. In this fault state, the robot arm is locked in position and cannot be moved by the operating room staff. A service engineer is required to run tests to assess any damage caused by the collision, replace/repair any damaged components, following which the robot arm can be reset to an operable mode. This servicing takes considerable time during which the robot arm cannot be used to perform surgery.

### SUMMARY OF THE INVENTION

According to an aspect of the invention, there is provided a control unit for controlling a surgical robot arm to enter a sleep mode, the surgical robot arm extending between a base and a terminal link via a series of articulated links, the base mounted on a support structure, the terminal link having an attachment for a surgical instrument, the control unit configured to: receive a command signal to enter the sleep mode, and respond by controlling the articulated links of the surgical robot arm to be driven to adopt a compact configuration relative to the support structure; receive sensor data from one or more sensors on the surgical robot arm; determine, from the received sensor data, a collision between the surgical robot arm and the support structure; respond to the determined collision by causing the surgical robot arm to change from the sleep mode to a locked mode, and controlling the articulated links to maintain their position at the time the locked mode was entered; whilst in the locked mode, receive a command signal to enter an unlocked mode, and respond by controlling the articulated links of the surgical robot arm to be driven to move the surgical robot arm away from the site of the determined collision ; and whilst in the unlocked mode, receive a command signal to enter the sleep mode, and respond by controlling the articulated links of the surgical robot arm to be driven to adopt the compact configuration.

The control unit may be configured to: receive sensed torque data from a torque sensor on the surgical robot arm; determine an excess torque from the sensed torque data by adjusting the received sensed torque data for expected torques at the torque sensor; compare the excess torque to a threshold excess torque; and determine a collision between the surgical robot arm and the support structure if the excess torque exceeds the threshold excess torque. The received sensed torque data may be for a joint about which a link adjacent to the terminal link of the surgical robot arm articulates.

The control unit may be configured to: receive sensed position data from a position sensor on the surgical robot arm; determine a sensed current position from the received sensed position data; determine a position error by comparing the sensed current position to an expected current position; compare the position error to a threshold position error; and determine a collision between the surgical robot arm and the support structure if the position error exceeds the threshold position error.

The received sensed position data may be for a joint about which a link adjacent to the terminal link of the surgical robot arm articulates.

The control unit may be configured to respond to the determined collision by controlling an alarm signal to be output. The control unit may be configured to control the surgical robot arm to output the alarm signal. The control unit may be configured to control the remote surgeon console to output the alarm signal.

In the compact configuration, the terminal link may nestle in a recess in the support structure. The support structure may comprise a height adjustable segment between the base of the robot arm and the recess. The control unit may be configured to, having caused the surgical robot arm to enter the locked mode in response to the determined collision, on subsequently receiving a command signal to reduce the height of the height adjustable segment so as to move the base of the surgical robot arm towards the recess, prevent the height adjustable segment being driven to move the base of the surgical robot arm towards the recess. The control unit may be configured to, having caused the surgical robot arm to enter the locked mode in response to the determined collision, on subsequently receiving a command signal to extend the height of the height adjustable segment so as to move the base of the surgical robot arm away from the recess, control the height adjustable segment to be driven to move the base of the surgical robot arm away from the recess.

The articulated links may be connected by joints, and the surgical robot arm comprise position sensors, each position sensor configured to measure the position of a joint. The control unit may be configured to: receive sensed position data from each of the position sensors; from the received sensed position data, determine whether the distance between the current position of the terminal link and the sleep position of the terminal link in the compact configuration is less than a threshold value; if the distance between the current position of the terminal link and the sleep position of the terminal link in the compact configuration is determined to be less than the threshold value, set a first threshold excess torque as the threshold excess torque; and if the distance between the current position of the terminal link and the sleep position of the terminal link in the compact configuration is determined to be greater than the threshold value, set a second threshold excess torque as the threshold excess torque, wherein the first threshold excess torque is less than the second threshold excess torque.

The articulated links may be connected by joints, and the surgical robot arm comprise position sensors, each position sensor for measuring the position of a joint. The control unit may be configured to: receive sensed position data from each of the position sensors; from the received sensed position data, determine whether the distance between the current position of the terminal link and the sleep position of the terminal link in the compact configuration is less than a threshold value; if the distance between the current position of the terminal link and the sleep position of the terminal link in the compact configuration is determined to be less than the threshold value, set a first threshold position error as the threshold position error; and if the distance between the current position of the terminal link and the sleep position of the terminal link in the compact configuration is determined to be greater than the threshold value, set a second threshold position error as the threshold position error, wherein the first threshold position error is less than the second threshold position error.

The support structure may be a cart.

The surgical robot arm may comprise a user input, the control unit being configured to receive the command signal to enter the sleep mode from the user input on the surgical robot arm.

The surgical robot arm may comprise a further user input, the control unit being configured to receive the command signal to enter the unlocked mode from the further user input on the surgical robot arm.

The control unit may be configured to control the surgical robot arm under the control of a remote surgeon console, the remote surgeon console comprising a user input, the control unit being configured to receive the command signal to enter the sleep mode from the user input on the remote surgeon console.

The remote surgeon console may comprise a further user input, the control unit being configured to receive the command signal to enter the unlocked mode from the further user input on the remote surgeon console.

In the sleep mode the surgical robot arm may be powered down, and the control unit may be configured to control the surgical robot arm to transition from the sleep mode to an off state in which it is powered off.

The compact configuration may be one which minimises the combined footprint of the surgical robot arm and support structure.

The compact configuration may be one in which the surgical robot arm is folded.

### BRIEF DESCRIPTION OF THE FIGURES

The present invention will now be described by way of example with reference to the accompanying drawings. In the drawings:

FIG. 1 illustrates a surgical robot system for performing a surgical procedure;

FIG. 2 illustrates a surgical robot mounted on a cart;

FIG. 3 illustrates a surgical robot;

FIG. 4 illustrates an instrument being positioned into engagement with a robot arm via an interface assembly;

FIG. 5 illustrates a collision between a surgical robot arm and a cart;

FIG. 6 illustrates a collision between a surgical robot arm and a cart;

FIG. 7 is a flowchart illustrating a control method for entering a sleep mode of the surgical robot arm; and

FIG. 8 illustrates an open configuration of the surgical robot arm.

### DETAILED DESCRIPTION

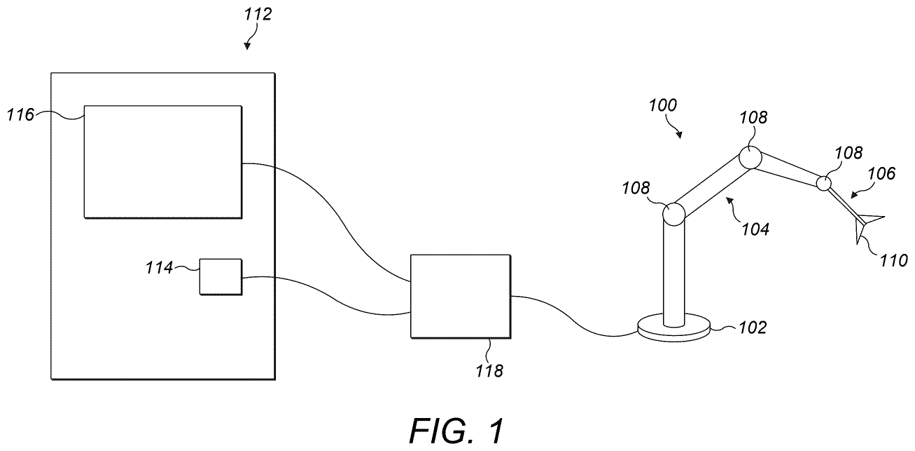

The following describes a method of controlling a surgical robot arm whilst entering a sleep mode. In particular, the control strategy following a collision between the surgical robot arm and its support structure whilst the surgical robot arm is being driven to adopt a predetermined sleep configuration is discussed. The surgical robot arm is mounted to the support structure. During surgery, the surgical robot arm has an instrument attached to its distal end, and operates under the control of a surgeon console via a control system as shown in FIG. 1 and discussed above.

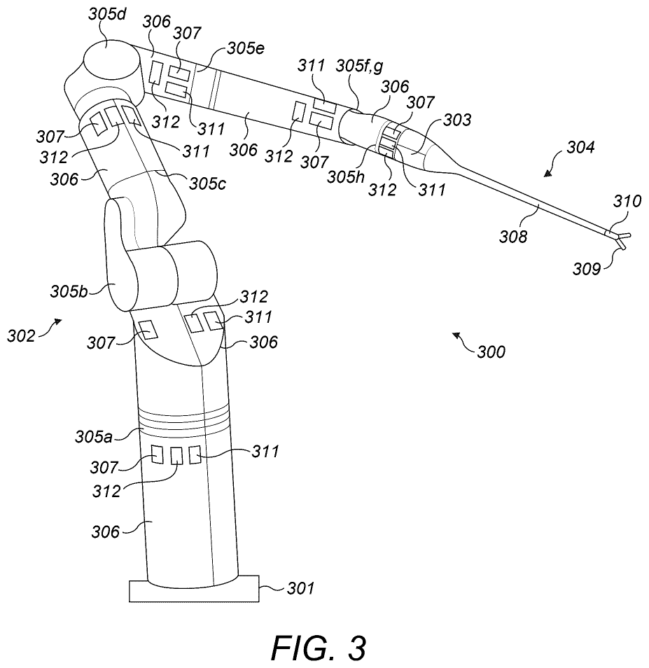

FIG. 3 illustrates an example robot 300. The robot comprises a base 301. A robot arm 302 extends from the base 301 of the robot to a terminal link 303 to which a surgical instrument 304 can be attached. The arm is flexible. It is articulated by means of multiple flexible joints 305 along its length. In between the joints are rigid arm links 306. The arm in FIG. 3 has eight joints. The joints include one or more roll joints (which have an axis of rotation along the longitudinal direction of the arm members on either side of the joint), one or more pitch joints (which have an axis of rotation transverse to the longitudinal direction of the preceding arm member), and one or more yaw joints (which also have an axis of rotation transverse to the longitudinal direction of the preceding arm member and also transverse to the rotation axis of a co-located pitch joint). In the example of FIG. 3: joints 305a, 305c, 305e and 305h are roll joints; joints 305b, 305d and 305f are pitch joints; and joint 305g is a yaw joint. Pitch joint 305f and yaw joint 305g have intersecting axes of rotation. The order of the joints from the base 301 to the terminal link 303 of the robot arm is thus: roll, pitch, roll, pitch, roll, pitch, yaw, roll. However, the arm could be jointed differently. For example, the arm may have fewer than eight or more than eight joints. The arm may include joints that permit motion other than rotation between respective sides of the joint, for example a telescopic joint.

The robot comprises a set of drivers 307. Each driver 307 has a motor which drives one or more of the joints 305. The terminal link 303 of the robot arm comprises a drive assembly interface for interfacing and driving a surgical instrument. The drive assembly interface comprises drive assembly interface elements which engage with corresponding instrument interface elements of an instrument interface of the surgical instrument. The drive assembly interface elements are driven by drivers 307. As the drive assembly interface elements move they move the instrument interface elements they are engaged with, thereby transferring drive from the drive assembly of the robot arm to the instrument interface of the instrument. Instrument 304 comprises a shaft 308 spanning between its proximal end which is attachable to the robot arm and its distal end which is attached to an end effector 309 via an articulation 310. The articulation 310 permits the end effector 309 to move relative to the shaft 308. The end effector 309 may comprise two end effector elements, each of which is independently rotatable relative to the articulation. Driving elements, such as cables, connect the instrument interface elements to the articulation and end effector through the shaft. As the instrument interface elements move, the driving elements move, which causes the articulation and/or end effector to move, thereby transferring drive to the distal end of the instrument.

The robot arm comprises a set of position sensors 311, one located at each joint for measuring the position of that joint, for example for measuring the joint angle of that joint. The robot arm also comprises a set of torque sensors 312, one located at each joint for measuring the torque about that joint. The sensed data from the position and torque sensors is sent to the control system 118.

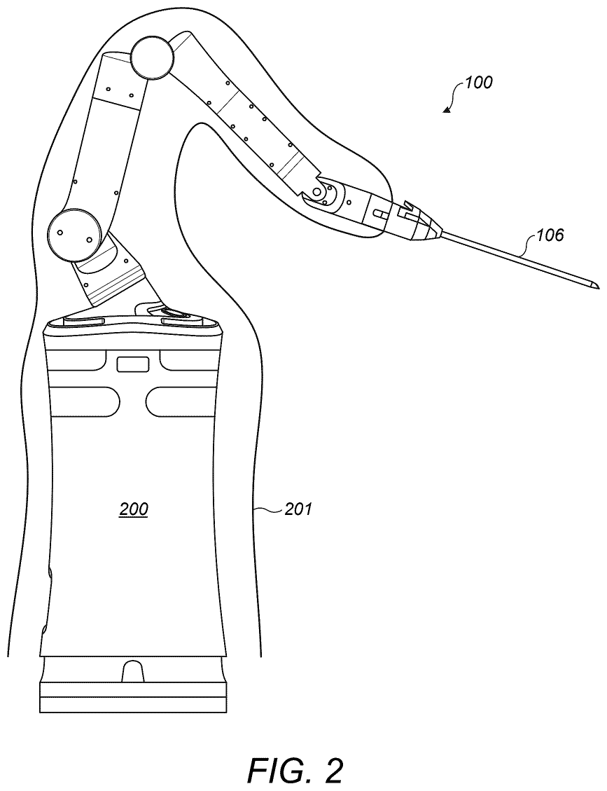

As shown in FIG. 2, during an operation, the surgical robot is shrouded in a sterile drape to provide a sterile barrier between the non-sterile surgical robot and the sterile operating environment. The surgical instrument is sterilised before being attached to the surgical robot. The sterile drape is typically constructed of a plastic sheet, for example made of polyester, polypropylene, polyethylene or polytetrafluoroethylene (PTFE). Suitably, the drape is flexible and/or deformable.

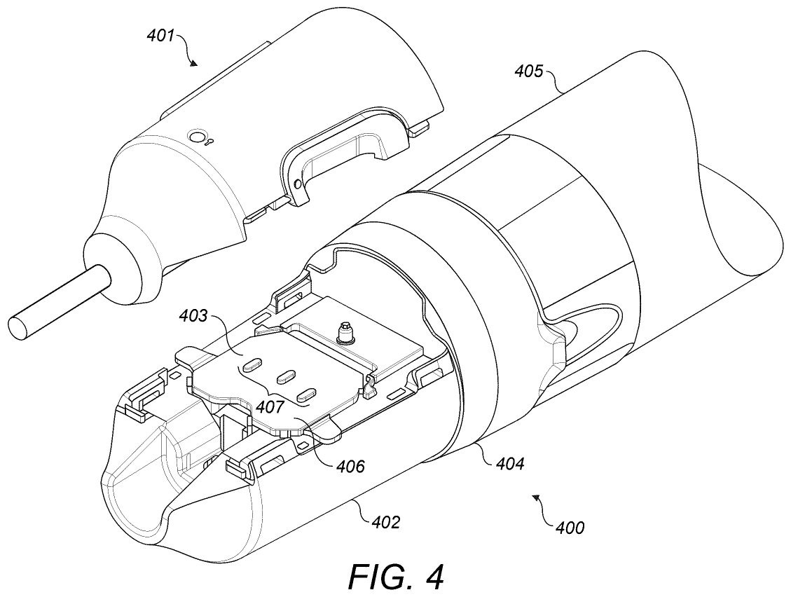

The sterile drape does not pass directly between the drive assembly interface and the instrument interface. FIG. 4 illustrates an interface assembly 400 which is attached to the drape for interfacing between the drive assembly interface and the instrument interface 401. The interface assembly 400 may have a sleeve or cup shape for pushing over the end of the robot arm. The exemplary interface assembly 400 comprises an interface structure 402 which engages the robot arm and the instrument so as to retain the interface structure to the robot arm and instrument respectively, and a removable drive interface structure 403. Drive interface structure 403 comprises a flexible material 406 and drive transfer elements 407 for interfacing between the instrument interface elements and the drive assembly interface elements. The interface assembly 400 may also include a moveable locking element 404 mounted on the interface structure 402 for locking the interface structure 402 to the robot arm 405. The drape may be attached to the locking element 404 or the interface structure 402. The interface structure 402 or the locking element 404 may be integrally formed with the drape. Alternatively, the interface structure 402 or the locking element 404 may be formed separately from the drape and subsequently attached to the drape. Either way, the interface structure 402 and the locking element 404 are sterile. One side of the interface structure 402 directly contacts the drive assembly interface. Another side of the interface structure 402 directly contacts the instrument interface. The interface structure 402 comprises a rim surrounding an aperture through which the drive assembly interface elements transfer drive to the interface assembly elements. The drive interface structure 403 is also sterile and comprises a flexible material 406 surrounded by a rigid rim. Rigid drive transfer elements 407 in the flexible material 406 interface the drive assembly interface elements on one side and the instrument interface elements on the other. Together, the flexible material 406 and drive transfer elements 407 provide a sterile barrier between the drive assembly interface elements and the instrument interface elements. Thus, the interface structure 402 and drive interface structure 403 together prevent the non-sterile drive assembly interface from directly touching the sterile instrument interface and hence maintain the sterile barrier between the surgical robot and the surgical instrument.

At the end of the operation, the instrument, drape and interface assembly are removed from the robot arm. The control system then receives a command signal from a user input to put the robot into a sleep mode. The user input may be on the robot arm itself, for example a button or switch on the robot arm. The user input may be on the surgeon console, for example a button, switch or pedal on the surgeon console. Several user inputs may be provided, for example one on the robot arm and another on the surgeon console, each of which can be actuated to generate a command signal to the control system to put the robot into sleep mode.

The control system responds to the command signal to put the robot arm into sleep mode by sending control signals to the drivers 307 of the robot arm to drive the joints 305 to move the robot arm into a predetermined sleep configuration. This predetermined sleep configuration is a compact configuration which minimises the combined footprint of the surgical robot arm and support structure upon which it is mounted. It is thereby the most suitable configuration for storing the robot arm and support structure when it is subsequently turned off. Suitably, in this compact configuration, the robot arm is folded and the terminal end of the robot arm slots into a recess in the support structure. This additionally provides protection of the drive assembly interface whilst the robot arm is stowed.

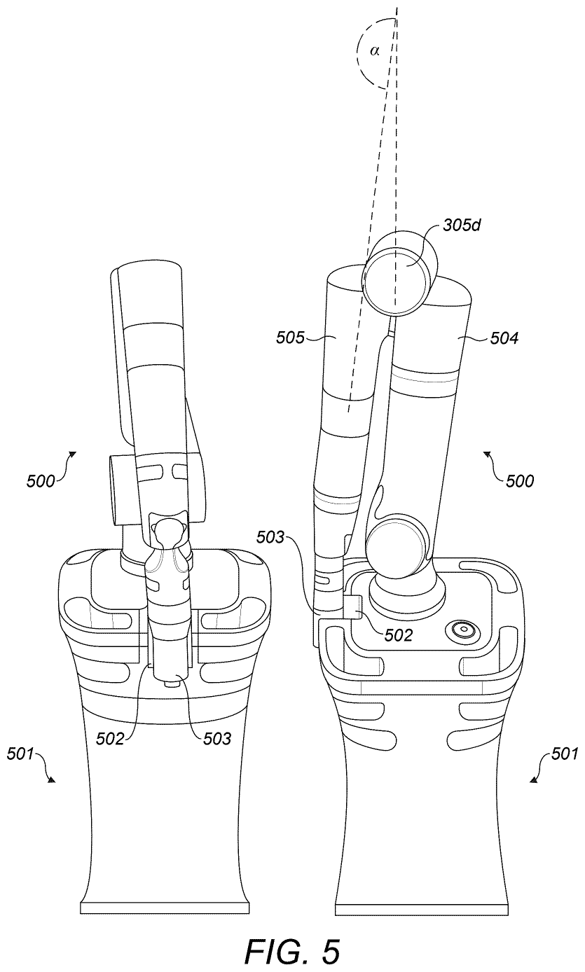

FIG. 5 illustrates a surgical robot arm 500 mounted on a cart 501. The robot arm is part way through being driven to adopt the compact configuration of the sleep mode. The robot arm is folded. The terminal end of the robot arm should nestle in the recess 502 in the cart 501. However, the interface assembly 503 has not been removed. The robot arm cannot therefore reach the final resting position of the terminal end of the robot arm in the recess 502. The robot arm continues to be driven to adopt the compact configuration, which results in a collision between the robot arm and the side of the cart.

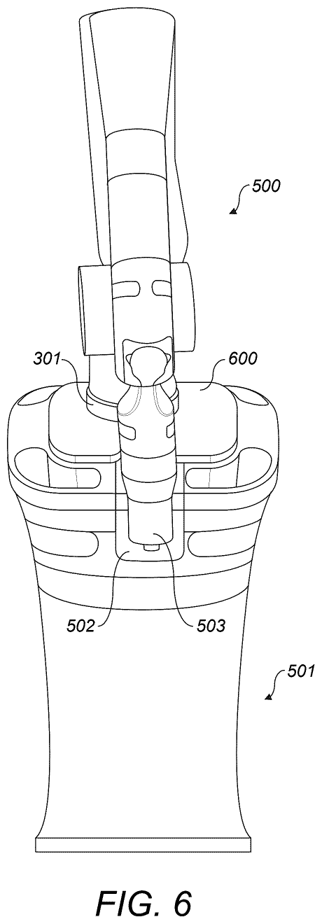

FIG. 6 illustrates another scenario in which the cart 501 comprises a height adjustable segment 600. The height adjustable segment 600 is between the base of the robot arm and the recess 502 in the cart. In this case, the predetermined sleep configuration comprises a compact configuration in which the robot arm is folded and the terminal end of the robot arm nestles into the recess 502 in the cart, and also in which the height adjustable segment 600 is lowered to its minimum height. Again, the interface assembly 503 has not been removed. The robot arm is first driven to adopt the folded position which it can do. Then the height adjustable segment 600 is driven to be lowered, so as to move the base of the surgical robot arm 301 towards the recess 502. The interface assembly 503 prevents the height adjustable segment 600 being lowered to its minimum height. The height adjustable segment 600 continues to be driven to adopt this configuration, which results in a collision between the robot arm and the base of the recess 502.

Other scenarios exist which result in a collision when the sleep mode is activated. For example, if the instrument or drape are not removed before the sleep mode is activated, then either of these may prevent the robot arm from being able to adopt its folded position nestled into the recess in the cart. As another example, if an object had been left in the recess then this would prevent the robot arm from being able to adopt its folded position nestled into the recess.

The control system receives sensor data from the joint position sensors 311 and joint torque sensors 312. The control system detects the collision from the position and/or torque sensor data. The control system sends command signals to the drivers 307 of the robot arm to drive the joints to adopt commanded joint positions in the sleep configuration. The control system may compare the commanded joint position of each joint to the sensed joint position of that joint, and determine a tracking error for that joint as the difference between the commanded and sensed joint position of the joint. If the tracking error of the joint exceeds a threshold, then the control system may determine a collision has occurred. The tracking error threshold may be in the range 0.02 to 0.06 radians. The tracking error threshold may be different for different joints. The control system may only determine a collision has occurred if the tracking error of a single specific joint exceeds the threshold. That specific joint may be the joint closest to the terminal end of the robot arm which rotates about an axis transverse to the longitudinal axis of either arm link it connects. That specific joint may be pitch joint 305f. The specific joint may be yaw joint 305g.

The control system may respond to the collision detection by causing the robot arm to enter a fault state. In the fault state, the robot arm is locked in position. In order to comply with regulations, that fault state cannot be exited by the operating room staff. A service engineer attends to the robot arm and performs tests to assess any damage, replace/repair any damaged components, following which the robot arm is reset to an operable mode.

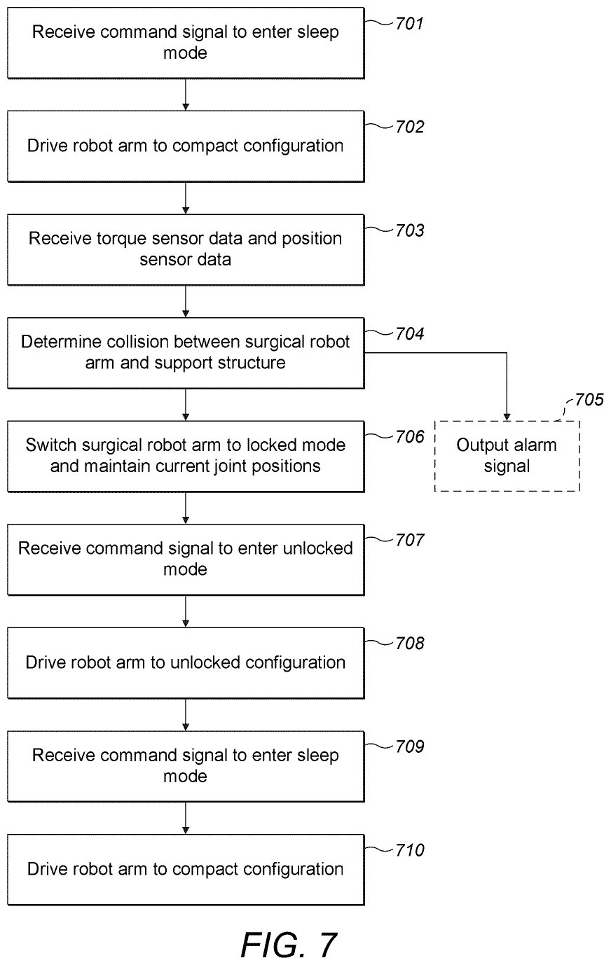

Alternatively, or in addition to the collision detection and fault state response described above, the control system may perform the following control method when entering the sleep mode which will be described with reference to FIG. 7.

At step 701, the control system receives a command signal to enter the sleep mode. As discussed above, this command signal may come from a user input at the robot arm, the surgeon console or elsewhere. At step 702, the control system responds to this command to enter the sleep mode by controlling the drivers 307 of the surgical robot arm to drive the robot arm joints 305 such that the surgical robot arm adopts a predetermined sleep configuration. This is a compact configuration relative to the support structure. The support structure may be a mobile cart. Alternatively, the support structure may be a non-mobile cart, the operating table, the patient bed, or other suitable chassis.

At step 703, the control system receives torque sensor data from the torque sensors 312 on the surgical robot arm and position sensor data from the position sensors 311 on the surgical robot arm as described above. At step 704, the control system determines a collision between the surgical robot arm and the support structure from the received sensor data.

The control system may determine a collision between the surgical robot arm and the support structure from the received torque sensor data. To do this, the control system may, for a robot arm joint, determine an excess torque by adjusting the received sensed torque data for expected torques at that torque sensor. The expected torques may include any one or combination of: torque due to gravitational forces acting on the joint, torque due to the joint being driven as commanded by the control system, and torque due to frictional and/or inertial forces acting on the joint. The excess torque may be determined by deducting the expected torques from the received torque sensor data. The control system then compares the excess torque to a threshold excess torque. The control system determines a collision between the surgical robot arm and the support structure if the excess torque exceeds the threshold excess torque.

The control system may only determine a collision has occurred if the excess torque of a single specific joint exceeds the threshold excess torque. That specific joint may be the joint closest to the terminal end of the robot arm which rotates about an axis transverse to the longitudinal axis of either arm link it connects. The specific joint may be a joint about which a link adjacent to the terminal link of the surgical robot arm articulates. That specific joint may be pitch joint 305f. The specific joint may be yaw joint 305g. The threshold excess torque may be in the range 1 Nm to 10Nm. The threshold excess torque may be in the range 0.5 Nm to 5 Nm. The threshold excess torque may be 1.5 Nm.

The control system may determine a collision between the surgical robot arm and the support structure from the received position sensor data. To do this, the control system may, for a joint, determine a sensed current position from the received sensed position data. The control system then determines a position error for that joint by comparing the commanded joint position of the joint to the sensed joint position of that joint, and determining a position error for that joint to be the difference between the commanded and sensed joint position of that joint. The control system then compares the position error to a threshold position error. The control system determines a collision between the surgical robot arm and the support structure if the position error exceeds the threshold position error.

The control system may only determine a collision has occurred if the position error of a single specific joint exceeds the threshold position error. That specific joint may be the joint closest to the terminal end of the robot arm which rotates about an axis transverse to the longitudinal axis of either arm link it connects. The specific joint may be a joint about which a link adjacent to the terminal link of the surgical robot arm articulates. That specific joint may be pitch joint 305f. The specific joint may be yaw joint 305g. The threshold position error may be in the range 0.005 radians to 0.1 radians. The threshold position error may be in the range 0.005 radians to 0.05 radians. The threshold position error may be 0.01 radians.

The method of FIG. 7 is much more sensitive than the collision detection and fault state response described above. The excess torque threshold which may be applied in the method of FIG. 7 is much lower than the excess torque threshold applied in the collision detection and fault state response. The threshold position error which may be applied in the method of FIG. 7 is much lower than the threshold position error applied in the collision detection and fault state response. No damage will have been inflicted on the robot arm at these thresholds, and as such, the robot arm can be returned to an operable state by the operating room staff and continue to be used. There is no need for the surgical robot to be serviced to ensure it is still safe to use.

In response to the detection of the collision, the control system may optionally at step 705 control an alarm signal to be output. This alarm signal may be audible and/or visual and/or haptic. The control system may control the surgical robot arm and/or support structure to output the alarm signal. The control system may additionally or alternatively control the surgeon console to output the alarm signal. For example, an audible alarm may be output from speakers on the surgical robot arm and/or support structure and/or surgeon console. A visual alarm such as a flashing light may be output from the surgical robot arm and/or support structure and/or surgeon console. A haptic alarm may also be output. For example, the robot arm may be driven to shake at the input that the user is actuating to put the robot arm into sleep mode. As another example, the surgeon's hand controllers at the surgeon console may be driven to shake.

In response to the detection of the collision, at step 706 the control system causes the surgical robot arm to transition from the sleep mode to a locked mode. The control system also causes the joints of the surgical robot arm to be locked in the position they had at the time the locked mode was entered. Whilst in the locked mode, the control system receives a command signal from a further user input at step 707 to enter an unlocked mode. That further user input may be on the robot arm itself, for example a button or switch on the robot arm. The further user input may be on the surgeon console, for example a button, switch or pedal on the surgeon console. Several further user inputs may be provided, for example one on the robot arm and another on the surgeon console, each of which can be actuated to generate a command signal to the control system to put the robot into an unlocked mode. The user input and further user input may be the same input.

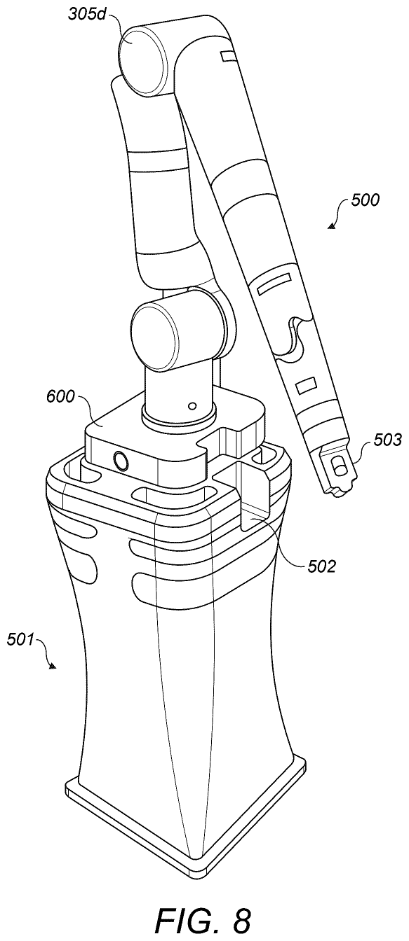

The control system responds to the command to enter the unlocked mode by, at step 708, controlling the drivers 307 to drive the joints 305 of the robot arm to adopt an unlocked configuration. This unlocked configuration is one in which the surgical robot arm is moved away from the site of the collision. The unlocked configuration is suitably an open configuration. In the open configuration, the robot arm may have been opened out of its folded position to a configuration in which a surgical instrument could be attached to the end of the robot arm. The unlocked configuration may be a compliant mode in which the robot arm can be moved compliantly, thereby enabling the operating room staff to move the robot arm away from the site of the collision. In a compliant mode, the control system responds to external forces applied to the arm by driving the joint motors to move the arm in the direction of the force. Thus, if a user pushes the robot arm, it will respond by moving in the direction it is pushed. An exemplary unlocked configuration is illustrated in FIG. 8. Once in the unlocked configuration, the operating room staff can resolve the cause of the collision. For example, they can remove the obstruction such as the interface assembly and/or instrument and/or drape from the robot arm in this unlocked configuration. They can access the recess 502 to remove any item that is blocking the terminal end of the robot arm from nestling in the recess 502.

Once the cause of the collision has been resolved, the operating room staff command the robot to transition to the sleep mode by actuating the user input. At the time this command is received, the surgical robot is in the unlocked mode. The control system receives this command to transition to the sleep mode at step 709. The control system responds by transitioning the surgical robot from the unlocked mode to the sleep mode by, at step 710, controlling the drivers 307 of the robot arm to drive the robot arm joints 305 such that the surgical robot arm moves to adopt the predetermined sleep configuration.

Once the robot arm has been successfully driven to the compact configuration at step 710 of FIG. 7, the control system may control the robot arm to be transitioned from the sleep mode in which it is powered down to an off state in which it is powered off. This transition to the off state may be in response to a command signal from a user input. Alternatively, this transition to the off state may be performed by the control system in response to sensing that the robot arm has successfully adopted the compact configuration.

As described with reference to step 706, whilst the surgical robot is in the locked mode, the surgical robot arm is locked in position. In the example in which the cart 501 comprises a height adjustable segment 600 between the base of the robot arm and the recess 502 in the cart, the control system restricts motion of the height adjustable segment during the locked mode. During the locked mode, the control system enables the height adjustable segment 600 to be extended such that the base of the robot arm moves away from the recess 502 in the cart. However, during the locked mode, the control system prevents the height adjustable segment 600 being lowered such that the base of the robot arm moves towards the recess 502 in the cart. Thus, if, whilst in the locked mode at step 706, the control system receives a command signal to reduce the height of the height adjustable segment so as to move the base of the surgical robot arm towards the recess, the control system does not carry out this command and prevents the driving mechanism of the height adjustable segment being driven as commanded. However, if, whilst in the locked mode at step 706, the control system receives a command signal to extend the height of the height adjustable segment so as to move the base of the surgical robot arm away from the recess, the control system does carry out this command by controlling the height adjustable segment to move accordingly. Once the control system has moved to step 708 in which the robot arm enters the unlocked mode, the control system no longer restricts motion of the height adjustable segment. Thus, in the unlocked mode, the control system will carry out a command signal to raise/extend or lower/retract the height adjustable segment.

Two control methods have been described above of how the control system responds to received sensor data whilst driving the robot arm to a compact configuration in the sleep mode: (i) the collision detection and fault state response method, and (ii) the control method described with reference to FIG. 7. One or the other of these control methods may be performed during the sleep mode. Alternatively, the control methods may be performed in parallel during the sleep mode. The collision detection and fault state response method may be employed in respect of one or more joints of the robot arm, whilst the control method of FIG. 7 is employed in respect of one or more other joints of the robot arm.

The control system may employ the collision and fault state response method during an initial stage of driving the robot arm to adopt the compact configuration, and the control method of FIG. 7 during a latter stage of driving the robot arm to adopt the compact configuration. Suitably, the control system employs the collision and fault state response method during the initial stage of driving the robot arm to adopt the compact configuration in respect of all the joints of the robot arm. In the latter stage of driving the robot arm to adopt the compact configuration, the control system employs the control method of FIG. 7 in respect of a single specific joint of the robot arm and the collision and fault state response method in respect of the remaining joints of the robot arm. That specific joint may be the joint closest to the terminal end of the robot arm which rotates about an axis transverse to the longitudinal axis of either arm link it connects. The specific joint may be a joint about which a link adjacent to the terminal link of the surgical robot arm articulates. That specific joint may be pitch joint 305f. The specific joint may be yaw joint 305g. The control system may move from the initial stage to the latter stage of driving the robot arm to adopt the compact configuration when the current configuration of the robot arm is at or approaching a configuration in which a collision could occur with the support structure.

This change from the initial to the latter stage of driving the robot arm with respect to the joint(s) which change from the collision and fault state response method to the control method of FIG. 7 may be implemented as follows. The control system carries out steps 701, 702 and 703 of the method of FIG. 7 as described above. Then, from the received sensed position data, the control system determines whether the distance between the current position of the terminal link and the sleep position of the terminal link in the compact configuration is less than a threshold value. That threshold value may be in the range 2 cm to 30 cm. The threshold value may be in the range 5 cm to 20 cm. The threshold value may be 10 cm. If the distance between the current position of the terminal link and the sleep position of the terminal link in the compact configuration is less than the threshold value, then the control system continues with the remainder of the steps described with respect to FIG. 7. If the distance between the current position of the terminal link and the sleep position of the terminal link in the compact configuration is greater than the threshold value, then the control system does not continue with the remainder of the steps described with respect to FIG. 7, but instead performs the collision and fault state response method for that joint(s).

The control system may determine whether the distance between the current position of the terminal link and the sleep position of the terminal link in the compact configuration is less than a threshold value by, for example:
(i) determining the current position of a joint about which the link adjacent to the terminal link of the surgical robot arm articulates, that current position being relative to the base of the surgical robot arm, that current position being determined from the received sensed position data for that joint and the known structure of the surgical robot arm,
(ii) calculating the distance between this current position of the joint and the sleep position of the joint relative to the base of the surgical robot arm in the compact configuration,
(iii) comparing this calculated distance to the threshold value, (iv) determining that the distance between the current position of the terminal link and the sleep position of the terminal link is less than the threshold value if the calculated distance is less than the threshold value, and
(v) determining that the distance between the current position of the terminal link and the sleep position of the terminal link is greater than the threshold value if the calculated distance is greater than the threshold value.

The control system may determine whether the distance between the current position of the terminal link and the sleep position of the terminal link in the compact configuration is less than a threshold value by, for example, measuring the joint angle of joint 305d. As the robot arm approaches the compact configuration the arm folds at joint 305d, as can be seen in FIGS. 5 and 8. The joint angle a of joint 305d between the arm links 504 and 505 it connects as measured from the straight configuration in which arm links 504 and 505 are colinear increases towards 180°. The control system may determine that the distance between the current position of the terminal link and the sleep position of the terminal link is less than the threshold value if the joint angle a of joint 305d is greater than a threshold joint angle. This threshold joint angle may be in the range 150° to 170°. This threshold joint angle may be in the range 160° to 165°.

Utilising the method of FIG. 7 enables the operating room staff to be alerted to a problem with driving the robot arm to the compact configuration in the sleep mode sooner than they are with the collision detection and fault state response method. Motion of the robot arm is locked sooner before the robot arm is exposed to high collision forces which may damage it. Thus, the robot arm does not need to be serviced for damage and recalibration. Instead, the problem can be recovered by the operating room staff by the robot arm being moved into a configuration in which the cause of the collision can be removed, and then commanding the robot arm to enter the sleep mode again.

As described above, the control system discussed herein connects the surgeon console to the surgical robot. The control system receives inputs from the surgeon input device(s) 114 and converts these to control signals to move the joints of the robot arm and instrument. The control system sends these control signals to the robot, where the corresponding joints are driven accordingly. The control system also receives sensor data from the position and torque sensors on the robot arm, and uses this data as described herein. The control system comprises a processor and memory. The memory stores in a non-transient way software that is executable by the processor to control the operation of: (i) the drivers 307 to cause the robot arm and instrument to operate in the manner described herein, (ii) the height adjustable segment to operate in the manner described herein, and (iii) the surgeon console to operate in the manner described herein. The control system may be located remote from the surgeon console and the surgical robot arm and support structure. The control system may be co-located with the surgeon console, the support structure or the surgical robotic arm. The control system may alternatively be a distributed control system which is located at a combination of any of the following: the surgeon console, the support structure, the robot arm, and a location remote from any of the surgeon console, support structure and robot arm.

The sleep mode referenced herein may be an inactive mode. The sleep mode is a lower power mode than active modes such as the surgical mode. Surgery cannot be performed in the sleep mode. Suitably, the sleep mode is not a compliant mode.

The applicant hereby discloses in isolation each individual feature described herein and any combination of two or more such features, to the extent that such features or combinations are capable of being carried out based on the present specification as a whole in the light of the common general knowledge of a person skilled in the art, irrespective of whether such features or combinations of features solve any problems disclosed herein, and without limitation to the scope of the claims. The applicant indicates that aspects of the present invention may consist of any such individual feature or combination of features. In view of the foregoing description it will be evident to a person skilled in the art that various modifications may be made within the scope of the invention.

1. A control unit configured to control a surgical robot arm to enter a sleep mode, the surgical robot arm extending between a base and a terminal link via a series of articulated links, the base mounted on a support structure, the terminal link having an attachment to hold a surgical instrument, the control unit configured to:
receive a command signal to enter the sleep mode, and respond by controlling the articulated links of the surgical robot arm to be driven to adopt a compact configuration relative to the support structure;
receive sensor data from one or more sensors on the surgical robot arm;
determine, from the received sensor data, a collision between the surgical robot arm and the support structure;
respond to the determined collision by causing the surgical robot arm to change from the sleep mode to a locked mode, and controlling the articulated links to maintain their position at the time the locked mode was entered;
whilst in the locked mode, receive a command signal to enter an unlocked mode, and respond by controlling the articulated links of the surgical robot arm to be driven to move the surgical robot arm away from the site of the determined collision; and
whilst in the unlocked mode, receive a command signal to enter the sleep mode, and respond by controlling the articulated links of the surgical robot arm to be driven to adopt the compact configuration.

2. The control unit as claimed in claim 1, configured to:
receive sensed torque data from a torque sensor on the surgical robot arm;
determine an excess torque from the sensed torque data by adjusting the received sensed torque data for expected torques at the torque sensor;
compare the excess torque to a threshold excess torque; and
determine a collision between the surgical robot arm and the support structure if the excess torque exceeds the threshold excess torque.

3. The control unit as claimed in claim 2, wherein the received sensed torque data is for a joint about which a link adjacent to the terminal link of the surgical robot arm articulates.

4. The control unit as claimed in claim 1, configured to:
receive sensed position data from a position sensor on the surgical robot arm;
determine a sensed current position from the received sensed position data;
determine a position error by comparing the sensed current position to an expected current position;
compare the position error to a threshold position error; and
determine a collision between the surgical robot arm and the support structure if the position error exceeds the threshold position error.

5. The control unit as claimed in claim 4, wherein the received sensed position data is for a joint about which a link adjacent to the terminal link of the surgical robot arm articulates.

6. The control unit as claimed in claim 1, configured to respond to the determined collision by controlling an alarm signal to be output.

7. The control unit as claimed in claim 6, configured to control the surgical robot arm to output the alarm signal.

8. The control unit as claimed in claim configured to control the surgical robot arm under the control of a remote surgeon console, the control unit configured to cause the remote surgeon console to output the alarm signal.

9. The control unit as claimed in claim 1, wherein in the compact configuration, the terminal link nestles in a recess in the support structure, and the support structure comprises a height adjustable segment between the base of the robot arm and the recess, the control unit configured to, having caused the surgical robot arm to enter the locked mode in response to the determined collision, on subsequently receiving a command signal to reduce the height of the height adjustable segment so as to move the base of the surgical robot arm towards the recess, prevent the height adjustable segment being driven to move the base of the surgical robot arm towards the recess.

10. The control unit as claimed in claim 1, wherein in the compact configuration, the terminal link nestles in a recess in the support structure, and the support structure comprises a height adjustable segment between the base of the robot arm and the recess, the control unit configured to, having caused the surgical robot arm to enter the locked mode in response to the determined collision, on subsequently receiving a command signal to extend the height of the height adjustable segment so as to move the base of the surgical robot arm away from the recess, control the height adjustable segment to be driven to move the base of the surgical robot arm away from the recess.

11. The control unit as claimed in claim 2, wherein the articulated links are connected by joints, the surgical robot arm comprising position sensors, each position sensor configured to measure the position of a joint, the control unit being configured to:
receive sensed position data from each of the position sensors;
from the received sensed position data, determine whether the distance between the current position of the terminal link and the sleep position of the terminal link in the compact configuration is less than a threshold value;
if the distance between the current position of the terminal link and the sleep position of the terminal link in the compact configuration is determined to be less than the threshold value, set a first threshold excess torque as the threshold excess torque; and
if the distance between the current position of the terminal link and the sleep position of the terminal link in the compact configuration is determined to be greater than the threshold value, set a second threshold excess torque as the threshold excess torque,
wherein the first threshold excess torque is less than the second threshold excess torque.

12. The control unit as claimed in claim 4, wherein the articulated links are connected by joints, the surgical robot arm comprising position sensors, each position sensor configured to measure the position of a joint, the control unit being configured to:
receive sensed position data from each of the position sensors;
from the received sensed position data, determine whether the distance between the current position of the terminal link and the sleep position of the terminal link in the compact configuration is less than a threshold value;
if the distance between the current position of the terminal link and the sleep position of the terminal link in the compact configuration is determined to be less than the threshold value, set a first threshold position error as the threshold position error; and
if the distance between the current position of the terminal link and the sleep position of the terminal link in the compact configuration is determined to be greater than the threshold value, set a second threshold position error as the threshold position error,
wherein the first threshold position error is less than the second threshold position error.

13. The control unit as claimed in claim 1, wherein the support structure is a cart.

14. The control unit as claimed in claim 1, wherein the surgical robot arm comprises a user input, the control unit being configured to receive the command signal to enter the sleep mode from the user input on the surgical robot arm.

15. The control unit as claimed in claim 14, wherein the surgical robot arm comprises a further user input, the control unit being configured to receive the command signal to enter the unlocked mode from the further user input on the surgical robot arm.

16. The control unit as claimed in claim 1, configured to control the surgical robot arm under the control of a remote surgeon console, the remote surgeon console comprising a user input, the control unit being configured to receive the command signal to enter the sleep mode from the user input on the remote surgeon console.

17. The control unit as claimed in claim 16, wherein the remote surgeon console comprises further user input, the control unit being configured to receive the command signal to enter the unlocked mode from the further user input on the remote surgeon console.

18. The control unit as claimed in claim 1, wherein in the sleep mode the surgical robot arm is powered down, the control unit configured to control the surgical robot arm to transition from the sleep mode to an off state in which it is powered off.

19. The control unit as claimed in claim 1, wherein the compact configuration is one which minimises the combined footprint of the surgical robot arm and support structure.

20. The control unit as claimed in claim 1, wherein the compact configuration is one in which the surgical robot arm is folded.
